(19) United States
(12) Patent Application Publication
(10) Pub. No.: US 2021/0043271 A1
(43) Pub. Date: Feb. 11, 2021

(54) COMPUTER-IMPLEMENTED METHODS OF ESTIMATING A PROBABILITY OF LIVE BIRTH IN AN INFERTILITY SUBJECT, COMPUTER-READABLE MEDIA, AND DIAGNOSTICS

(71) Applicant: Women & Infants Hospital Of Rhode Island, Providence, RI (US)

(72) Inventors: Ruben Alvero, Los Altos, CA (US); Shunping Wang, Providence, RI (US); Yen-Tsung Huang, Nankang, Taipei (TW); Yi (Jamie) Zhang, Sunnyvale, CA (US)

(73) Assignee: Women & Infants Hospital Of Rhode Island, Providence, RI (US)

(21) Appl. No.: 16/980,001

(22) PCT Filed: Mar. 8, 2019
(86) PCT No.: PCT/US19/21370

§ 371 Date: Sep. 11, 2020

Related U.S. Application Data
(60) Provisional application No. 62/642,811, filed on Mar. 14, 2018.

(51) Int. Cl. G16B 5/20; G01N 33/76; G16H 50/50

(52) U.S. Cl. CPC G16B 5/20; G01N 33/76; G01N 2800/367; G01N 2333/59; G16H 50/50

(57) ABSTRACT

A method of estimating a probability of live birth in an infertility subject includes: receiving a parameter set including an anti-Mllerian hormone (AMH) value and a follicle-stimulating hormone (FSH) value, the AMH and FSH values generated using one or more samples from the infertility subject; adding a constant to the AMH value and the FSH value to generate an increased AMH value and an increased FSH value; transforming the increased AMH value and the increased FSH value to produce an augmented parameter set including a logarithmic AMH value and a logarithmic FSH value; and solving a generalized additive mixed model for a probability of live birth in the infertility subject based the augmented parameter set, wherein the generalized additive mixed model was previously backfit with a data set of subject data including the augmented parameter set for each infertility subject in the data set.

### CROSS-REFERENCE TO RELATED APPLICATION

This application claims the benefit of priority to U.S. Provisional Patent Application Ser. No. 62/642,811, filed Mar. 14, 2018. The entire content of this application is hereby incorporated by reference herein.

### BACKGROUND OF THE INVENTION

Anti-Müllerian hormone (AMH) and follicle-stimulating hormone (FSH) are used to assess ovarian reserve in infertility patients.

### SUMMARY OF THE INVENTION

One aspect of the invention provide a computer-implemented method of estimating a probability of live birth in an infertility subject. The computer-implemented method includes: receiving a parameter set including an anti-Müllerian hormone (AMH) value and a follicle-stimulating hormone (FSH) value, the AMH and FSH values generated using one or more samples from the infertility subject; adding a constant to the AMH value and the FSH value to generate an increased AMH value and an increased FSH value; transforming the increased AMH value and the increased FSH value to produce an augmented parameter set including a logarithmic AMH value and a logarithmic FSH value; and solving a generalized additive mixed model for a probability of live birth in the infertility subject based the augmented parameter set, wherein the generalized additive mixed model was previously backfit with a data set of subject data including the augmented parameter set for each infertility subject in the data set, thereby estimating the probability of live birth in the infertility subject.

This aspect of the invention can have a variety of embodiments. The generalized additive mixed model can be a penalized log-likelihood generalized additive mixed model. The generalized additive mixed model can be a semiparametric regression generalized additive mixed model. The generalized additive mixed model can be a penalized spline generalized additive mixed model.

The parameter set and the augmented parameter set can further include one or more demographics. The one or more demographics can include one or more selected from the group consisting of: age and parity.

The one or more samples can be selected from the group consisting of: blood, blood serum, blood plasma, and urine.

The AMH value can be determined using enzyme-linked immunosorbent assay (ELISA). The FSH value can be determined using an assay selected from the group consisting of: a radioimmunoassay (RIA) and chemiluminescence immunoassay.

The infertility subject can be a human female. The human female can be selected from the group consisting of females of advanced maternal age, females having oocyte-related infertility and females having low ovarian reserve.

Another aspect of the invention provides a non-transitory computer-readable medium containing program instructions executable by a processor. The computer readable medium includes program instructions to implement a computer-implemented method of as described herein.

This aspect of the invention can have a variety of embodiments. The infertility subject can be a human female. The human female can be selected from the group consisting of females of advanced maternal age, females having oocyte-related infertility and females having low ovarian reserve.

Another aspect of the invention provides a method of diagnosing and treating an infertility subject. The method includes: obtaining a parameter set including an anti-Müllerian hormone (AMH) value and a follicle-stimulating hormone (FSH) value, the AMH and FSH values generated using one or more samples from the infertility subject; providing the parameter set as an input to either a computer-implemented method as described herein or a computer executing the program instructions of a non-transitory computer-readable medium as described herein; receiving a probability of live birth in the infertility subject; and treating the infertility subject based on the probability.

This aspect of the invention can have a variety of embodiments. The infertility subject can be a human female. The human female can be selected from the group consisting of females of advanced maternal age, females having oocyte-related infertility and females having low ovarian reserve.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 is a CONSORT diagram for a data preparation process for analysis according to an embodiment of the invention.

FIG. 2 depicts estimated marginal dose-responsive relationships between estimated live birth rates and (A) AMH and (B) FSH for patients of age 30, 35, 37, and 40 years old by generalized additive mixed models.

FIG. 3 depicts estimated joint effects of AMH and FSH on live birth rates by two-dimensional generalized additive mixed models for patients from different age groups. Panels depict the following age groups: (A) 30 years old, (B) 35 years old, (C) 37 years old, and (D) 40 years old. Green indicates a good prognosis (AMH ≥1 ng/ml & FSH <10 mIU/ml). Yellow indicates a poor prognosis (AMH <1 ng/ml & FSH ≥10 mIU/ml). Red represents a FSH reassuring group (AMH <1 ng/ml & FSH <10 mIU/ml). Grey represents an AMH reassuring group (AMH ≥1 ng/ml & FSH >10 mIU/ml).

FIG. 4 depicts a computer-implemented method 400 of estimating a probability of live birth in an infertility subject according to an embodiment of the invention.

FIG. 5 depicts a method 500 of diagnosing and treating an infertility subject according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

The terms “subject” and “patient” are used interchangeably and include any live-bearing member of the class mammalia, including humans, domestic and farm animals, and zoo, sports or pet animals, such as mouse, rabbit, pig, sheep, goat, cattle, horses and higher primates. In certain embodiments, the subject/patient is a human female. In certain embodiments, the subject/patient is a human female selected from the group consisting of females of advanced maternal age, females having oocyte-related fertility and females having low ovarian reserve.

As used herein, the term “advanced maternal age” as it relates to humans refers to a woman who is 34 years of age or older.

As used herein, the term “oocyte-related infertility” as it relates to humans refers to an inability to conceive after one year of unprotected intercourse which is not caused by an anatomical abnormality (e.g., blocked oviduct) or pathological condition (e.g., uterine fibroids, severe endometriosis, Type II diabetes, polycystic ovarian disease).

As used herein, the term “low ovarian reserve” as it relates to humans refers to a woman who exhibits a circulating Follicle Stimulating Hormone (FSH) level greater than 15 mIU/ml in a “day 3 FSH test,” as described in Scott et al., Fertility and Sterility, 1989 51:651-4, or a circulating Anti-Müllerian Hormone (AMH) level less than 0.6 ng/ml, or an antral follicle count less than 7 as measured by ultrasound.

### DETAILED DESCRIPTION OF THE INVENTION

In women undergoing evaluation for infertility, ovarian reserve testing with anti-Müllerian hormone (AMH) and follicle stimulating hormone (FSH) provides important prognostic information regarding reproductive outcomes.

AMH is a peptide hormone produced by granulosa cells of early antral follicles and can be collected at any point during a woman's menstrual cycle. FSH is a hormone produced by the anterior pituitary.

Both markers are affected by a woman's age: AMH decreases as age increases, while FSH increases as age increases.

Although AMH and FSH are generally accepted as useful in predicting response to ovarian stimulation, existing evidence is controversial regarding the utility of both markers for the prediction of live birth, with current studies limited by small sample sizes and stringent inclusion criteria that limits their external validity. The question, therefore, of which ovarian reserve marker is a better predictor of live birth remains unanswered, leaving infertility specialists with limited evidence to guide their treatment decisions.

Clinicians additionally often encounter a discrepancy between the two markers—a situation that can affect the interpretation of a woman's likelihood of live birth. Leader et al. showed a frequency of AMH and FSH discordance of as many as 1 in 5 evaluations for female infertility. B. Leader et al., “High frequency of discordance between antimüllerian hormone and follicle-stimulating hormone levels in serum from estradiol-confirmed days 2 to 4 of the menstrual cycle from 5,354 women in U.S. fertility centers,” 98 Fertil. Steril. 1037-42 (2012). In a small retrospective study, having an elevated FSH (>10 mIU/ml), but reassuring AMH (>0.6 ng/ml) was found to be significantly associated with higher oocyte yield, greater number of day 3 embryos, and lower cycle cancellation rates compared to women with random AMH levels <0.6 ng/ml. Clinical pregnancy rate among this group was likewise higher, but the difference was not statistically significant. E. Buyuk et al., “Random anti-Müllerian hormone (AMH) is a predictor of ovarian response in women with elevated baseline early follicular follicle-stimulating hormone levels”, 95 Fertil. Steril. 2369-72 (2011). Gleicher et al. similarly reported that among 115 female infertility patients with discordant AMH and FSH (normal age specific AMH with abnormal FSH), oocyte yield was diminished compared to their AMH/FSH concordant counterparts (normal age specific AMH and FSH). N. Gleicher et al., “Toward a better understanding of functional ovarian reserve: AMH (AMHo) and FSH (FSHo) hormone ratios per retrieved oocyte”, 97 J. Clin. Endocrinol. Metab. 995-1004 (2010). Still, when discordant results are encountered, there is a paucity of data regarding the prognostic relationship between AMH and FSH.

Aspects of the invention utilize generalized additive mixed models to address this clinical problem by providing a more accurate estimate of probability of live birth in an infertility subject. Embodiments of the invention can utilize these probabilities to inform clinical decisions regarding treatment of the infertility subject.

### Computer-Implemented Method of Estimating Probability of Live Birth in an Infertility Subject

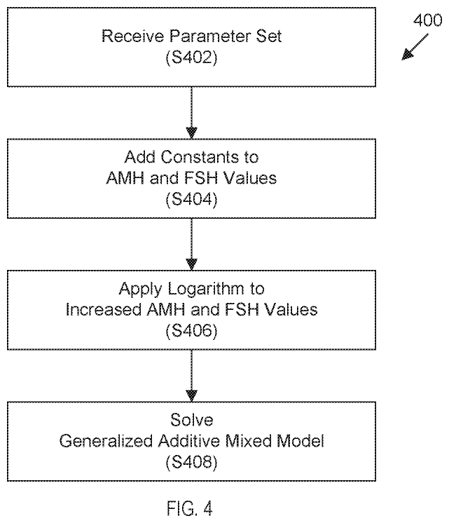

Referring now to FIG. 4, one aspect of the invention provides a computer-implemented method 400 of estimating a probability of live birth in an infertility subject.

In step S402, a parameter set is received. The parameter set can include an anti-Müllerian hormone (AMH) value and a follicle-stimulating hormone (FSH) value generated using one or more samples from the infertility subject such as blood, blood serum, blood plasma, urine, and the like. The AMH value can be generated using a variety of techniques such as an enzyme-linked immunosorbent assay (ELISA). The FSH value can be generated using a variety of techniques such as a radioimmunoassay (RIA), a chemiluminescence immunoassay, and the like.

The AMH value and the FSH value can be generated locally (e.g., within a clinic's or hospital's laboratory) or can be obtained from an external clinical laboratory service such as, e.g., Quest Diagnostics Incorporated of Madison, N.J.

In step S404, a constant is added to the AMH value and the FSH value to generate an increased AMH value and an increased FSH value. The constant can be a positive real number (e.g., a positive rational number, a natural number, and the like).

In step S406, the increased AMH value and the increased FSH value are transformed using a logarithmic function to produce an augmented parameter set including a logarithmic AMH value and a logarithmic FSH value.

The parameter set and the augmented parameter set can include additional data beyond AMH and FSH values such as, e.g., age, parity (the number of times a female has given birth), and the like.

In step S408, a generalized additive mixed model (GAMM) is solved for a probability of live birth in the infertility subject based the augmented parameter set. The GAMM can have been previously backfit with a data set of subject data including the augmented parameter set for each infertility subject in the data set. For example, the GAMM can be backfit using the R™ programming language and software environment for statistical computing and graphics provided by the R Foundation for Statistical Computing. The GAMM can be a penalized log-likelihood GAMM, a semiparametric regression GAMM, a penalized spline GAMM, and the like.

### Method of Diagnosing and Treating an Infertility Subject

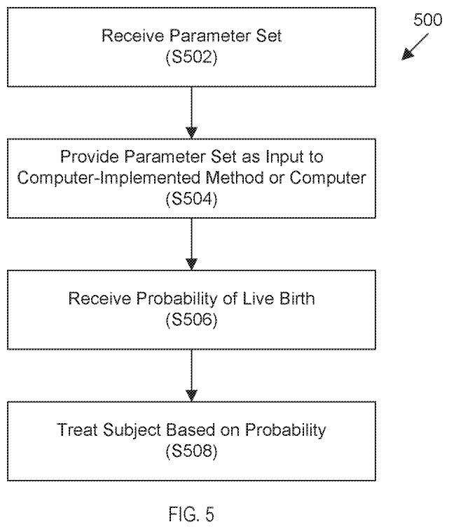

Referring now to FIG. 5, another aspect of the invention provides a method 500 of diagnosing and treating an infertility subject.

In step S502, a parameter set is received. The parameter set can include an anti-Müllerian hormone (AMH) value and a follicle-stimulating hormone (FSH) value generated using one or more samples from the infertility subject, e.g., as described in the context of step S402.

In step S504, the parameter set is provided as an input to a computer-implemented method as described herein (e.g., computer-implemented method 400) or a computer executing program instructions (e.g., stored in computer-readable media). For example, the parameter set can be input by a healthcare professional, e.g., using a computer (e.g., a desktop, a laptop, a tablet, a smartphone, and the like). In another embodiment, the parameter set can be provided electronically by a laboratory or the methods described herein can be performed by the laboratory after generating the parameter set.

In step S506, a probability of live birth in the infertility subject is received. The probability can be expressed in a variety of forms such as percentages, ratios, fractions, and the like. The probability can be provided electronically (e.g., as an input to the infertility subject's electronic medical record, via e-mail, via a secure portal), via a printed report, aurally, and the like.

In step S508, the subject is treated based on the probability. A variety of infertility treatments include in vitro fertilization, ovarian hyperstimulation, controlled ovarian hyperstimulation, natural cycle in vitro fertilization, final maturation induction, transvaginal oocyte retrieval, egg and sperm preparation, co-inoculation, embryo culture, adjunctive medication, cycle-stimulation therapies, FSH therapies, microdose gonadotropin-releasing hormone antagonist (GnRHa) flare therapies, antagonist (e.g., GnRHant) therapies, augmented intracytoplasmonic sperm injection, mitochondrial augmented intracytoplasmic sperm injection and related female germline stem cell treatments (e.g., the AUGMENT℠, OVAPRIME℠ and OVATURE℠ treatments offered by OvaScience, Inc. of Waltham, Mass.), adoption, and the like.

For example, various probability ranges can indicate use of a particular treatment. In one embodiment, infertility subjects having a probability of live birth of 10% or greater can be treated using better individualization in selection of stimulation protocols. For example, a poor responder as predicted by embodiments of the invention can be prescribed a micro-dose flare, which is less suppressive and may allow for better response of follicles. Similarly, a polycystic ovary syndrome (PCOS) patient with likelihood of exuberant response could be prescribed an antagonist protocol.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like), non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### WORKING EXAMPLE

### Patient Selection

EIVF™ is an electronic medical record software for clinical IVF settings designed by PracticeHwy.com (Dallas, Tex.). Applicant obtained a dataset including 144,044 fresh cycles from 60 centers in the United States from 2000 to 2016. Evaluation of this comprehensive de-identified dataset was determined to be exempt by the Women & Infants Institutional Review Board (Women & Infants Hospital of Rhode Island).

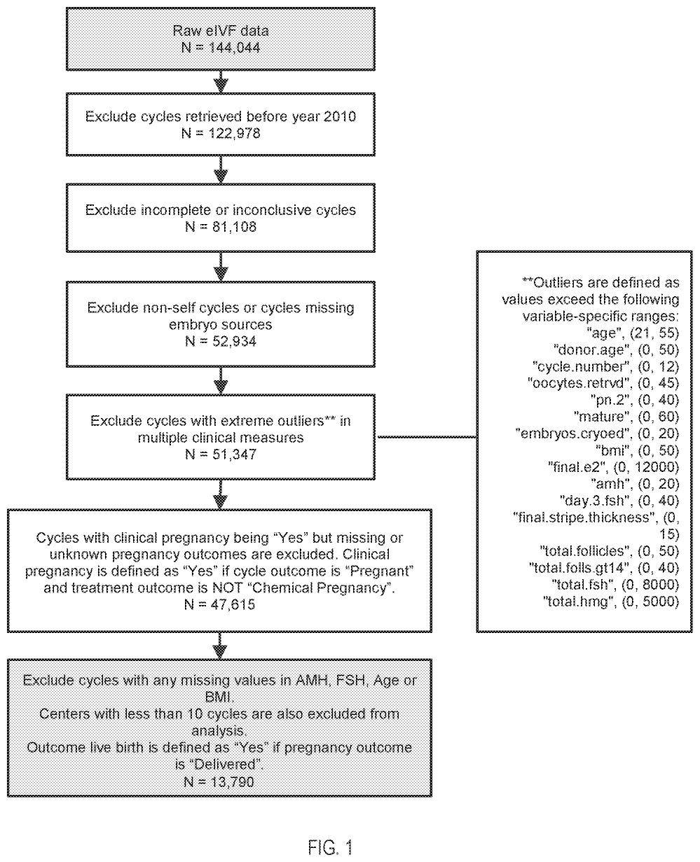

FIG. 1 shows a CONSORT diagram for data processing. Applicant excluded cycles that were incomplete, were non-autologous donor cycles, had unknown or missing cycle information, or contained outlier variables. Centers with less than 10 cycles were also excluded. Following application of these exclusion criteria, only 47,615 cycles remained in the dataset. Of note, because AMH has only been adopted in clinical use in the past few years, most cycles before 2010 were excluded because of missing AMH values. Thus, the final dataset contained 13,790 autologous IVF cycles with known AMH, FSH, and confirmed determination of live birth.

The 13,790 cycles included for analysis were further subdivided into four groups using AMH=1.0 ng/ml and FSH=10.0 mIU/ml as cutoff values for normal/reassuring testing. Groups I and II represent a patient population with concordance between their AMH and FSH results. Group I included cycles from all good prognosis patients with AMH greater than or equal to 1.0 ng/ml and FSH less than 10 mIU/ml. Group II included cycles from patients considered poor responders based on AMH less than 1.0 ng/ml and FSH greater than or equal to 10 mIU/ml. Groups III and IV represent a patient population with discordance between their ovarian reserve markers. Group III included the cycles with AMH less than 1.0 ng/ml with FSH less than 10 mIU/ml, while Group IV included cycles with AMH greater than or equal to 1.0 ng/ml with FSH greater than or equal to 10 mIU/ml (see Table 1 below). The primary outcome of interest was live birth per cycle initiated. Statistical Analysis

Generalized additive mixed models (GAMM) were used to investigate the nonlinear fixed effects of AMH and FSH on live birth rate using penalized spline, while adjusting for the random effects of centers. AMH and FSH levels were transformed into log-scale before fitting the models because of their highly skewed distributions in the sample, and a small value, 0.7 was added to AMH and FSH levels before transformation to avoid taking logarithm of zero. GAMM were fit to delineate the marginal effects of AMH and FSH on live birth rate, adjusting for age. The joint effects of AMH and FSH were further characterized using two-dimensional spline under GAMM. The two-dimensional splines, with AMH-by-FSH interaction and without, were both explored to investigate the joint effects of AMH and FSH. All models were fitted through maximizing a penalized log-likelihood using RTM package mgcv. Based on the fitted models, Applicant was able to predict the probability of live birth for a patient given one's AMH, FSH and age. To visualize the dose-response relationship of AMH and/or FSH with respect to the probability of live birth, Applicant plotted predicted probabilities given the corresponding AMH and FSH under each model.

### Results

Table 1 presents the baseline characteristics of the four groups based on the previously defined cutoffs. Table 2 provides the p-values shown as superscripts the “Live Birth (%)” row.

TABLE 1
Demographic Characteristics and Live Birth Rates of All Four Groups

| | |
|---|---|
| Concordant | Discordant |

| | | Group I: | Group II: | Group III: | Group IV: |
|---|---|---|---|---|---|
| | | Good | Poor | Reassuring | Reassuring |
| | | Prognosis | Prognosis | FSH | AMH |
| | | (AMH ≥1 & | (AMH <1 & | (AMH <1 & | (AMH ≥1 & |
| | All | FSH <10) | FSH ≥10) | FSH <10) | FSH ≥10) |
| N | 13790 | 7997 | 1717 | 3271 | 805 |

| | Mean (SD) | | Mean (SD) | | Mean (SD) | | Mean (SD) | | Mean (SD) | | P-values |
|---|---|---|---|---|---|---|---|---|---|---|---|
| Age | 35.4 | (4.7) | 34.1 | (4.5) | 38.0 | (4.1) | 37.3 | (4.3) | 35.6 | (4.4) | <0.001 |
| BMI | 25.9 | (6.0) | 26.0 | (6.0) | 25.1 | (5.2) | 26.6 | (6.3) | 24.4 | (4.9) | <0.001 |
| # of embryos | 1.7 | (1.1) | 1.8 | (1.0) | 1.5 | (1.3) | 1.8 | (1.2) | 1.9 | (1.2) | <0.001 |
| transferred | | | | | | | | | | | |
| E2 | 2261 | (1485) | 2676 | (1556) | 1370 | (973) | 1690 | (1151) | 2240 | (1243) | <0.001 |
| FSH | 7.6 | (3.8) | 6.2 | (2.0) | 14.0 | (4.4) | 6.6 | (2.2) | 12.2 | (3.1) | <0.001 |
| (mIU/ml) | | | | | | | | | | | |
| AMH | 2.4 | (2.7) | 3.6 | (2.9) | 0.4 | (0.3) | 0.5 | (0.3) | 2.3 | (1.7) | <0.001 |
| (ng/ml) | | | | | | | | | | | |

| | | | | | | |
|---|---|---|---|---|---|---|
| Live Birth | 23.5% | 29.1%^{a, b, c} | 12.8%^{d, e} | 15.4%^{f} | 22.7% | <0.001 |
| (%) | | | | | | |

The live-birth rate for good-prognosis patients (Group I) was significantly higher than patients with poor-prognosis (Group II) (29.1% vs 12.8%; p <0.05). Among the two discordant groups, patients with reassuring AMH (Group IV) had significantly a higher live-birth rate compared to patients with reassuring FSH (Group III) (22.7% vs 15.4%, p <0.05).

TABLE 2
p-Values Between Groups

| | | |
|---|---|---|
| a | Group I vs. Group II | <0.001 |
| b | Group I vs. Group III | <0.001 |
| c | Group I vs. Group IV | <0.001 |
| d | Group II vs. Group III | <0.013 |
| e | Group II vs. Group IV | <0.001 |
| f | Group III vs. Group IV | <0.001 |

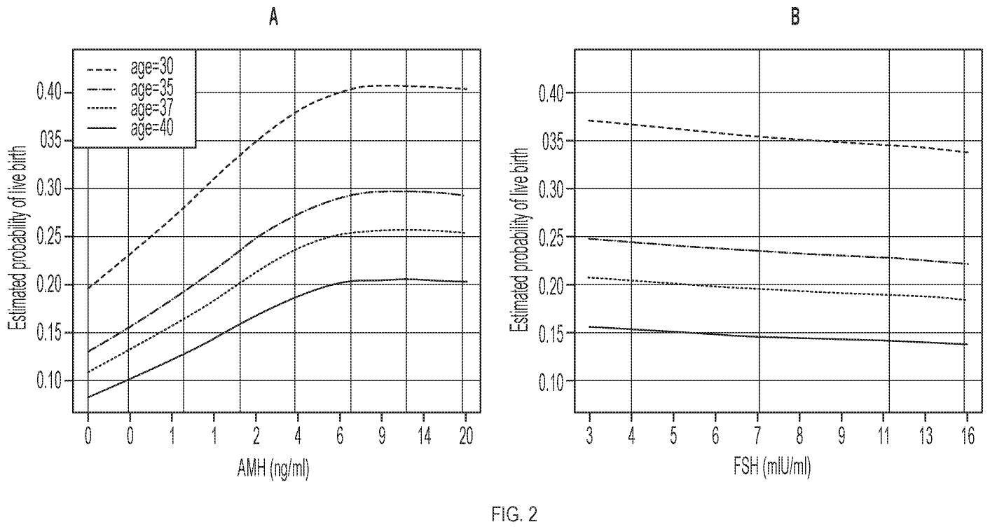

FIG. 2, Panels A and B show the GAMM established to predict the live-birth rate using AMH and FSH respectively among patients of age 30, 35, 37, and 40 years old. Among all ages examined for AMH, there was a positive dose-response relationship between AMH and probability of live birth (FIG. 2, Panel A). Similarly, among all ages examined for FSH, there was a negative dose-response relationship between FSH and live birth (FIG. 2, Panel B), although not as significant as AMH As AMH approached 6 ng/ml across all ages, there was a plateau in the estimated likelihood of live birth.

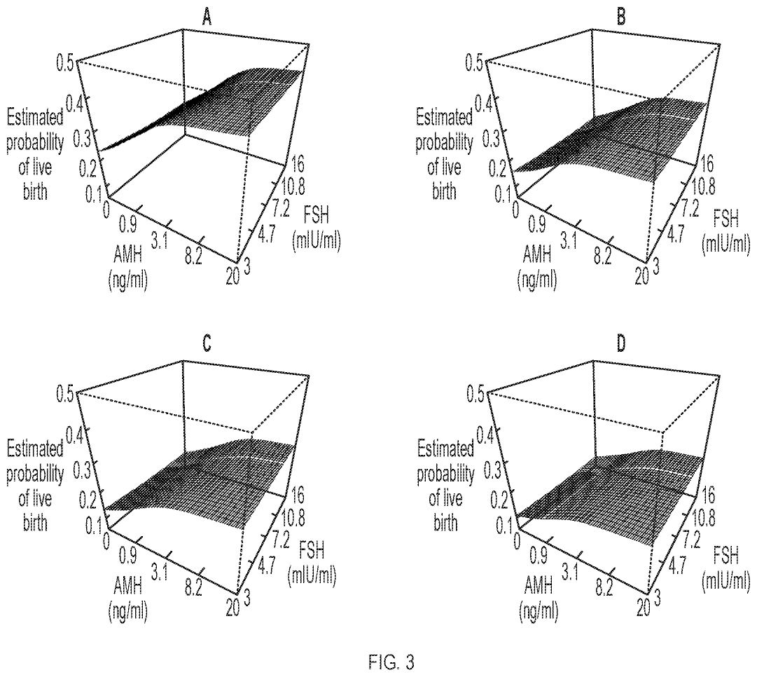

FIG. 3 demonstrates a model for the joint effect of AMH and FSH on live-birth rate. The two horizontal axes represent AMH and FSH values evenly spaced on log-scale, and the vertical axis indicates the estimated live-birth rates based on two-dimensional GAMM. The predicted birth rates for patients with age 30, 35, 37, and 40 years old are shown in FIG. 3, Panels A, B, C, and D, respectively. Consistent with the prior trend, the estimated probability of live birth decreases as age increases, given the same AMH and FSH. Within each figure panel of specified age, the predicted live-birth probability ascends rapidly with AMH when AMH is less than 8.2 ng/ml for fixed FSH. In comparison, for any given AMH value, the estimated live-birth probability only decreases slightly as FSH increases from the lowest truncated value to the highest. In other words, the joint effect of AMH and FSH is dominated by that of AMH. The three-dimensional graphs provide a comprehensive visualization of dose-response relationship between any combination of AMH, FSH, and live-birth rate. The joint effect analysis indicates that AMH is a more-reliable predictor of live birth rate than FSH. Particularly in the discordant groups, a reassuring AMH (grey region) suggests a better likelihood of live birth compared to reassuring FSH (red region). Consistent with the trend observed in marginal models, higher AMH has a positive effect on live-birth success rate while higher FSH and age demonstrate negative effects.

### Discussion

To Applicant's knowledge, this is the first comprehensive analysis of the clinical utility of AMH and FSH using statistically robust modeling with a sample size close to 14,000 cycles, with live birth as the primary outcome. AMH and FSH provide valuable prognostic clinical information prior to an IVF cycle start. Applicant's data suggest that although both markers confer some prognostic value to the prediction of live birth, AMH is superior to FSH among all age groups. This is suggested by two principal findings in FIG. 3. First, irrespective of FSH value, a low AMH confers a lower likelihood of live birth among young patients. This live-birth likelihood is even lower for older patients with low AMH. Additionally, in patients with high FSH, a high AMH rescues live birth probability (i.e., >20%) across all age groups. Both findings suggest AMH is the more important determinant of pregnancy outcome than FSH.

The study also suggests live-birth rates are highest in good prognosis cycles (Group I) and lowest in poor prognosis cycles (Group II). Prediction of cycle success is more difficult when AMH and FSH are discordant. In the 13,964 cycles analyzed, AMH and FSH levels were discordant (Group III: AMH >1 ng/ml and FSH >10 mIU/ml and Group IV: AMH<1 ng/ml and FSH<10 mIU/ml) in 30% of cycles, compared to a 20% discordance between AMH and FSH in over 5,300 women reported by Leader et al. Although the study of Leader et al. clearly describes a high rate of discordance between AMH and FSH, it is limited by the absence of clinical outcomes. In Applicant's study, a reassuring AMH predicted a higher live-birth rate among discordant cycles, likewise suggesting that a normal AMH is a better clinical predictor of cycle success when AMH and FSH are discordant.

Conventionally, logistic regression models, consisting of the first-order main effects of clinical measures, are used to investigate the clinical utility of AMH and FSH in predicting IVF success rate. These parametric approaches make several assumptions about the data, such as underlying linear relationship and normally distributed errors between the predictors and outcomes, which may not accurately reflect the nature of the clinical measures. In this study, Applicant utilized a semiparametric regression modeling—penalized spline regression—to reduce the assumed linear relationship between predictors and outcome. The piecewise continuous polynomials, or splines, when combined with mathematical penalization, provide a superior overall fit of the data compared to a conventional parametric approach. In addition, because the study sample was pooled from 26 IVF centers across the U.S., Applicant also adjusted for the center-level heterogeneity by including random intercept effects for each center in all models.

Applicant's results suggest a nonlinear relationship of AMH and live birth rate among all ages. Once AMH levels reach a certain threshold, the live-birth rate plateaus and further increases in AMH do not significantly increase the likelihood of live birth. Similarly, FSH demonstrates a nonlinear relationship with live birth rate; i.e., once FSH levels increase to a certain threshold, live birth rates decline for patients of all ages. These nonlinear relationships between AMH, FSH, and live birth importantly suggest that the ovarian reserve markers are associated with live birth in an age-dependent manner. The statistical approach used in Applicant's study to evaluate AMH and FSH is flexible in characterizing non-linear dose-response relationship between predictors and outcomes and, thus, provides an alternative analysis tool that could have been neglected in existing literature.

The marginal dose-response relationship of AMH or FSH with live birth rate (FIG. 2, Panels A and B), however, should be interpreted with certain caveats. For example, the marginal effect of AMH did not account for the effect contributed by FSH, and the high correlation of AMH and FSH may exert an undue influence, i.e., confounding on the AMH-live birth association. To address this issue, Applicant further characterized the joint effect of AMH and FSH using GAMM with two-dimensional splines to investigate the effect of one marker by adjusting for the other one. Applicant also explored the possibility of expanding the above models to include potential confounder BMI, which resulted in very similar results. Applicant applied the above prediction models to an internal dataset from patients to test the model validity. The Receiver Operating Curve area under curve calculation equaled 0.67, suggesting age, AMH, and FSH alone are perhaps not sufficient to accurately predict the IVF success rate. In conclusion, the ovarian-reserve markers AMH and FSH are both associated with live-birth probability, although AMH appears to be a stronger predictor especially in situations of discordant results. Prediction models that incorporate these markers in addition to other patient demographics and treatment response information are needed to provide accurate prognostic guidance for infertility specialists to facilitate patient counseling.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method of estimating a probability of live birth in an infertility subject, the computer-implemented method comprising:
receiving a parameter set comprising:
an anti-Müllerian hormone (AMH) value; and
a follicle-stimulating hormone (FSH) value;
the AMH and FSH values generated using one or more samples from the infertility subject;
adding a constant to the AMH value and the FSH value to generate an increased AMH value and an increased FSH value;
transforming the increased AMH value and the increased FSH value to produce an augmented parameter set comprising:
a logarithmic AMH value; and
a logarithmic FSH value; and
solving a generalized additive mixed model for a probability of live birth in the infertility subject based the augmented parameter set, wherein the generalized additive mixed model was previously backfit with a data set of subject data including the augmented parameter set for each infertility subject in the data set;
thereby estimating the probability of live birth in the infertility subject.

2. The computer-implemented method of claim 1, wherein the generalized additive mixed model is a penalized log-likelihood generalized additive mixed model.

3. The computer-implemented method of claim 1, wherein the generalized additive mixed model is a semiparametric regression generalized additive mixed model.

4. The computer-implemented method of claim 1, wherein the generalized additive mixed model is a penalized spline generalized additive mixed model.

5. The computer-implemented method of claim 1, wherein the parameter set and the augmented parameter set further comprise:
one or more demographics.

6. The computer-implemented method of claim 5, wherein the one or more demographics include one or more selected from the group consisting of: age and parity.

7. The computer-implemented method of claim 1, wherein the one or more samples are selected from the group consisting of: blood, blood serum, blood plasma, and urine.

8. The computer-implemented method of claim 1, wherein the AMH value is determined using enzyme-linked immunosorbent assay (ELISA).

9. The computer-implemented method of claim 1, wherein the FSH value is determined using an assay selected from the group consisting of: a radioimmunoassay (RIA) and chemiluminescence immunoassay.

10. The computer-implemented method of claim 1, wherein the infertility subject is a human female.

11. The computer-implemented method of claim 10, wherein the human female is selected from the group consisting of females of advanced maternal age, females having oocyte-related infertility and females having low ovarian reserve.

12. A non-transitory computer-readable medium containing program instructions executable by a processor, the computer readable medium comprising program instructions to implement the computer-implemented method of claim 1.

13. The non-transitory computer-readable medium of claim 12, wherein the infertility subject is a human female.

14. The non-transitory computer-readable medium of claim 13, wherein the human female is selected from the group consisting of females of advanced maternal age, females having oocyte-related infertility and females having low ovarian reserve.

15. A method of diagnosing and treating an infertility subject, the method comprising:
obtaining a parameter set comprising:
an anti-Müllerian hormone (AMH) value; and
a follicle-stimulating hormone (FSH) value; the AMH and FSH values generated using one or more samples from the infertility subject;
providing the parameter set as an input to a computer executing the program instructions of the non-transitory computer-readable medium 12;
receiving a probability of live birth in the infertility subject; and
treating the infertility subject based on the probability.

16. The method of claim 15, wherein the infertility subject is a human female.

17. The method of claim 16, wherein the human female is selected from the group consisting of females of advanced maternal age, females having oocyte-related infertility and females having low ovarian reserve.
